(19) United States
(12) Patent Application Publication
(10) Pub. No.: US 2005/0033397 A1
(43) Pub. Date: Feb. 10, 2005

(54) Low cost electrical stimulation and shock devices manufactured from conductive loaded resin-based materials

(75) Inventor: Thomas Aisenbrey, Littleton, CO (US)

Correspondence Address: GEORGE O. SAILE, 28 DAVID AVENUE, POUGHKEEPSIE, NY, 12603, US

(73) Assignee: Integral Technologies, Inc.

(21) Appl. No.: 10/897,277

(22) Filed: Jul. 22, 2004

Related U.S. Application Data
(60) Provisional application No. 60/492,348, filed on Aug. 4, 2003.

(51) Int. Cl. A61N 1/04

(52) U.S. Cl. 607/142

(57) ABSTRACT

Electrical stimulators and shocking devices are formed of a conductive loaded resin-based material. The conductive loaded resin-based material comprises micron conductive powder(s), conductive fiber(s), or a combination of conductive powder and conductive fibers in a base resin host. The percentage by weight of the conductive powder(s), conductive fiber(s), or a combination thereof is between about 20% and 50% of the weight of the conductive loaded resin-based material. The micron conductive powders are formed from non-metals, such as carbon, graphite, that may also be metallic plated, or the like, or from metals such as stainless steel, nickel, copper, silver, that may also be metallic plated, or the like, or from a combination of non-metal, plated, or in combination with, metal powders. The micron conductor fibers preferably are of nickel plated carbon fiber, stainless steel fiber, copper fiber, silver fiber, or the like.

This Patent Application claims priority to the U.S. Provisional Patent Application 60/492,348, filed on Aug. 4, 2004, which is herein incorporated by reference in its entirety.

This Patent Application is a Continuation-in-Part of INT01-002CIP, filed as U.S. patent application Ser. No. 10/309,429, filed on Dec. 4, 2002, also incorporated by reference in its entirety, which is a Continuation-in-Part application of docket No. INT01-002, filed as U.S. Pat. application Ser. No. 10/075,778, filed on Feb. 14, 2002, which claimed priority to U.S. Provisional Patent Applications Ser. No. 60/317,808, filed on Sep. 7, 2001, Ser. No. 60/269,414, filed on Feb. 16, 2001, and Ser. No. 60/268,822, filed on Feb. 15, 2001.

### BACKGROUND OF THE INVENTION

(1) Field of the Invention

This invention relates to electrical stimulation and shock devices and, more particularly, to electrical stimulation and shock devices molded of conductive loaded resin-based materials comprising micron conductive powders, micron conductive fibers, or a combination thereof, homogenized within a base resin when molded. This manufacturing process yields a conductive part or material usable within the EMF or electronic spectrum(s).

(2) Description of the Prior Art

Electrical stimulators and shocking devices are used in the art to transfer electrical energy directly into the body of a person or an animal. Electrical stimulation can be used for therapeutic applications, such as muscle or nerve stimulation, to aid in the healing process. Alternatively, electrical impulses are used in a defibrillator or a pace maker to restore proper heart rhythm in cases where the heart has ceased to beat or is beating out of proper rhythm. More aggressive application of electrical stimulus can result in actual electrical shock transmission. This approach is used in cattle prods and shock collars to inflict momentary pain for animal training or control. Stun guns or Taser guns can inflict physical and psychological pain and/or loss of muscular control and are used in law enforcement situations to subdue individuals.

Several prior art inventions relate to electrical stimulation and shock devices. U.S. Patent Publication US 2003/0004558 A1 to Gadsby teaches a disposable medical electrode intended for high energy stimulation utilizing a an electrically conductive, carbon filled polymer electrode. U.S. Patent Publication US 2001/0004238 A1 to Gerig teaches an animal shock collar with low impedance transformer for use in animal behavior modification systems. U.S. Patent Publication U.S. 2003/0165041 A1 to Stethem teaches a personal defense device or baton that has a flashlight and a shock device incorporated within. U.S. Patent Publication US 2003/0041768 to Rastegar teaches non-lethal deployable bullets capable of delivering a dose of tranquilizer, pepper spray, or an electronic shock to the intended target.

U.S. Patent Publication US 2004/0000274 A1 to James teaches an electrode for an animal training device that utilizes a flat electrode rather that the prong type. This invention also teaches that conductive polymers may be employed for the various surface conductors. U.S. Patent Publication US 2003/0094113 A1 to Warnagiris et al teaches a neuromuscular disrupter gun for delivery of a projectile containing a charged capacitor to a target. The internal capacitor comprises a metal foil and a liquid for the dielectric in the projectile. U.S. Patent Publication US 2003/0137795 A1 to Buening et al teaches a stun glove for directing an electric shock to an assailant. PVC is used as an insulator material. U.S. Pat. 6,095,148 to Shastri et al teaches neuronal stimulation using electrically conducting polymers utilizing a method of implanting seeds of the conductive polymer and applying electrical stimulation to surrounding tissue to encourage cell regeneration.

U.S. Patent Publication US 2003/0200830 A1 to Marmaropoulos et al teaches a wearable garment with medical electrode/sensors utilizing a medical sensor formed of flexible conductive silicone and “hook and loop” type fasteners also made of a conductive plastic material. U.S. Patent Publication US 2004/004341 A1 to Truckai et al teaches an electrosurgical tape able to deliver RF energy to target organs thereby causing tissue surface to shrink, coagulate, ablate or create lesions. The tape comprises conductive filaments in a biodegradable polymer and an insulative material exterior. U.S. Patent Publication US 2003/0158593 A1 to Heilman et al teaches a support garment for an external wearable defibrillator utilizing electrically conductive cloth for better electrical connection between the therapy electrodes and the patient's skin. The electrically conductive cloth taught in this invention comprises a metal mesh cloth covered in part by silver. U.S. Pat. No. 5,354,328 to Doan et al teaches a patch electrode for use with an implantable defibrillator utilizing a wire mesh as the conductor and a plastic or silicone material as the insulator.

### SUMMARY OF THE INVENTION

A principal object of the present invention is to provide an effective electrical stimulator or shock device.

A further object of the present invention is to provide a method to form an electrical stimulator or shock device.

A further object of the present invention is to provide an electrical stimulator or shock device molded of conductive loaded resin-based materials.

A yet further object of the present invention is to provide an electrical stimulator or shock device molded of conductive loaded resin-based material where the characteristics of the device can be altered or the visual characteristics can be altered by forming a metal layer over the conductive loaded resin-based material.

A yet further object of the present invention is to provide methods to fabricate an electrical stimulator or shock device from a conductive loaded resin-based material incorporating various forms of the material.

A yet further object of the present invention is to provide a method to fabricate an electrical stimulator or shock device from a conductive loaded resin-based material where the material is in the form of a fabric.

In accordance with the objects of this invention, an electrical stimulation device is achieved. The device comprises an electrical energy source and an electrode electrically connected to the electrical energy source. The electrode comprises a conductive loaded, resin-based material comprising conductive materials in a base resin host.

Also in accordance with the objects of this invention, an electrical stimulation device is achieved. The device comprises an electrical energy source, an electrode, and a conductive path between the electrical energy source and the electrode. The electrode and the conductive path comprise a conductive loaded, resin-based material comprising conductive materials in a base resin host.

Also in accordance with the objects of this invention, a method to form an electrode for an electrical stimulation device is achieved. The method comprises providing a conductive loaded, resin-based material comprising conductive materials in a resin-based host. The conductive loaded, resin-based material is molded into an electrode.

### BRIEF DESCRIPTION OF THE DRAWINGS

In the accompanying drawings forming a material part of this description, there is shown:

FIGS. 1a through 1d illustrate several preferred embodiments of the present invention showing electrical stimulation devices, such as defibrillator devices, comprising a conductive loaded resin-based material.

FIG. 2 illustrates a first preferred embodiment of a conductive loaded resin-based material wherein the conductive materials comprise a powder.

FIG. 3 illustrates a second preferred embodiment of a conductive loaded resin-based material wherein the conductive materials comprise micron conductive fibers.

FIG. 4 illustrates a third preferred embodiment of a conductive loaded resin-based material wherein the conductive materials comprise both conductive powder and micron conductive fibers.

FIGS. 5a and 5b illustrate a fourth preferred embodiment wherein conductive fabric-like materials are formed from the conductive loaded resin-based material.

FIGS. 6a and 6b illustrate, in simplified schematic form, an injection molding apparatus and an extrusion molding apparatus that may be used to mold electrical stimulation and shock devices of a conductive loaded resin-based material.

FIG. 7 illustrates a fifth preferred embodiment of the present invention showing an animal training or control collar device comprising conductive loaded resin-based material. FIG. 8 illustrates a sixth preferred embodiment of the present invention showing a cattle prodding device comprising conductive loaded resin-based material.

FIG. 9 illustrates a seventh preferred embodiment of the present invention showing a law enforcement, electric baton device comprising conductive loaded resin-based material.

FIG. 10 illustrates an eighth preferred embodiment of the present invention showing an electric stun gun device comprising conductive loaded resin-based material.

FIG. 11 illustrates a ninth preferred embodiment of the present invention showing an electric Taser gun device comprising conductive loaded resin-based material.

### DESCRIPTION OF THE PREFERRED EMBODIMENTS

This invention relates to electrical stimulation or shock devices molded of conductive loaded resin-based materials comprising micron conductive powders, micron conductive fibers, or a combination thereof, homogenized within a base resin when molded.

The conductive loaded resin-based materials of the invention are base resins loaded with conductive materials, which then makes any base resin a conductor rather than an insulator. The resins provide the structural integrity to the molded part. The micron conductive fibers, micron conductive powders, or a combination thereof, are homogenized within the resin during the molding process, providing the electrical continuity.

The conductive loaded resin-based materials can be molded, extruded or the like to provide almost any desired shape or size. The molded conductive loaded resin-based materials can also be cut, stamped, or vacuumed formed from an injection molded or extruded sheet or bar stock, over-molded, laminated, milled or the like to provide the desired shape and size. The thermal or electrical conductivity characteristics of electrical shock, stimulation, or stun devices fabricated using conductive loaded resin-based materials depend on the composition of the conductive loaded resin-based materials, of which the loading or doping parameters can be adjusted, to aid in achieving the desired structural, electrical or other physical characteristics of the material. The selected materials used to fabricate the electrical stimulation or shock devices are homogenized together using molding techniques and or methods such as injection molding, over-molding, thermo-set, protrusion, extrusion or the like. Characteristics related to 2D, 3D, 4D, and 5D designs, molding and electrical characteristics, include the physical and electrical advantages that can be achieved during the molding process of the actual parts and the polymer physics associated within the conductive networks within the molded part(s) or formed material(s).

The use of conductive loaded resin-based materials in the fabrication of electrical shock, stimulation, or stun devices significantly lowers the cost of materials and the design and manufacturing processes used to hold ease of close tolerances, by forming these materials into desired shapes and sizes. The electrical shock, stimulation, or stun devices can be manufactured into infinite shapes and sizes using conventional forming methods such as injection molding, over-molding, or extrusion or the like. The conductive loaded resin-based materials, when molded, typically but not exclusively produce a desirable usable range of resistivity from between about 5 and 25 ohms per square, but other resistivities can be achieved by varying the doping parameters and/or resin selection(s).

The conductive loaded resin-based materials comprise micron conductive powders, micron conductive fibers, or any combination thereof, which are homogenized together within the base resin, during the molding process, yielding an easy to produce low cost, electrically conductive, close tolerance manufactured part or circuit. The micron conductive powders can be of carbons, graphites, amines or the like, and/or of metal powders such as nickel, copper, silver, or plated or the like. The use of carbons or other forms of powders such as graphite(s) etc. can create additional low level electron exchange and, when used in combination with micron conductive fibers, creates a micron filler element within the micron conductive network of fiber(s) producing further electrical conductivity as well as acting as a lubricant for the molding equipment. The micron conductive fibers can be nickel plated carbon fiber, stainless steel fiber, copper fiber, silver fiber, or the like, or combinations thereof. The structural material is a material such as any polymer resin. Structural material can be, here given as examples and not as an exhaustive list, polymer resins produced by GE PLASTICS, Pittsfield, Mass., a range of other plastics produced by GE PLASTICS, Pittsfield, Mass., a range of other plastics produced by other manufacturers, silicones produced by GE SILICONES, Waterford, N.Y., or other flexible resin-based rubber compounds produced by other manufacturers.

The resin-based structural material loaded with micron conductive powders, micron conductive fibers, or in combination thereof can be molded, using conventional molding methods such as injection molding or over-molding, or extrusion to create desired shapes and sizes. The molded conductive loaded resin-based materials can also be stamped, cut or milled as desired to form create the desired shape form factor(s) of the heat sinks. The doping composition and directionality associated with the micron conductors within the loaded base resins can affect the electrical and structural characteristics of the electrical stimulation or shock devices and can be precisely controlled by mold designs, gating and or protrusion design(s) and or during the molding process itself. In addition, the resin base can be selected to obtain the desired thermal characteristics such as very high melting point or specific thermal conductivity.

A resin-based sandwich laminate could also be fabricated with random or continuous webbed micron stainless steel fibers or other conductive fibers, forming a cloth like material. The webbed conductive fiber can be laminated or the like to materials such as Teflon, Polyesters, or any resin-based flexible or solid material(s), which when discretely designed in fiber content(s), orientation(s) and shape(s), will produce a very highly conductive flexible cloth-like material. Such a cloth-like material could also be used in forming electrical stimulation or shock devices that could be embedded in a person's clothing as well as other resin materials such as rubber(s) or plastic(s). When using conductive fibers as a webbed conductor as part of a laminate or cloth-like material, the fibers may have diameters of between about 3 and 12 microns, typically between about 8 and 12 microns or in the range of about 10 microns, with length(s) that can be seamless or overlapping.

The conductive loaded resin-based material of the present invention can be made resistant to corrosion and/or metal electrolysis by selecting micron conductive fiber and/or micron conductive powder and base resin that are resistant to corrosion and/or metal electrolysis. For example, if a corrosion/electrolysis resistant base resin is combined with stainless steel fiber and carbon fiber/powder, then a corrosion and/or metal electrolysis resistant conductive loaded resin-based material is achieved. Another additional and important feature of the present invention is that the conductive loaded resin-based material of the present invention may be made flame retardant. Selection of a flame-retardant (FR) base resin material allows the resulting product to exhibit flame retardant capability. This is especially important in electrical stimulation or shock devices applications as described herein.

The homogeneous mixing of micron conductive fiber and/or micron conductive powder and base resin described in the present invention may also be described as doping. That is, the homogeneous mixing converts the typically non-conductive base resin material into a conductive material. This process is analogous to the doping process whereby a semiconductor material, such as silicon, can be converted into a conductive material through the introduction of donor/acceptor ions as is well known in the art of semiconductor devices. Therefore, the present invention uses the term doping to mean converting a typically non-conductive base resin material into a conductive material through the homogeneous mixing of micron conductive fiber and/or micron conductive powder into a base resin.

As an additional and important feature of the present invention, the molded conductor loaded resin-based material exhibits excellent thermal dissipation characteristics. Therefore, electrical stimulation or shock devices manufactured from the molded conductor loaded resin-based material can provide added thermal dissipation capabilities to the application. For example, heat can be dissipated from electrical devices physically and/or electrically connected to electrical stimulation or shock devices of the present invention.

As a significant advantage of the present invention, electrical shock, stimulation, or stun devices constructed of the conductive loaded resin-based material can be easily interfaced to an electrical circuit. In one embodiment, a wire can be attached to a conductive loaded resin-based electrical stimulation or shock device via a screw that is fastened to the device. For example, a simple sheet-metal type, self tapping screw can, when fastened to the material, achieves excellent electrical connectivity via the conductive matrix of the conductive loaded resin-based material. To facilitate this approach a boss may be molded into the conductive loaded resin-based material to accommodate such a screw. Alternatively, if a solderable screw material, such as copper, is used, then a wire can be soldered to the screw that is embedded into the conductive loaded resin-based material. In another embodiment, the conductive loaded resin-based material is partly or completely plated with a metal layer. The metal layer forms excellent electrical conductivity with the conductive matrix.

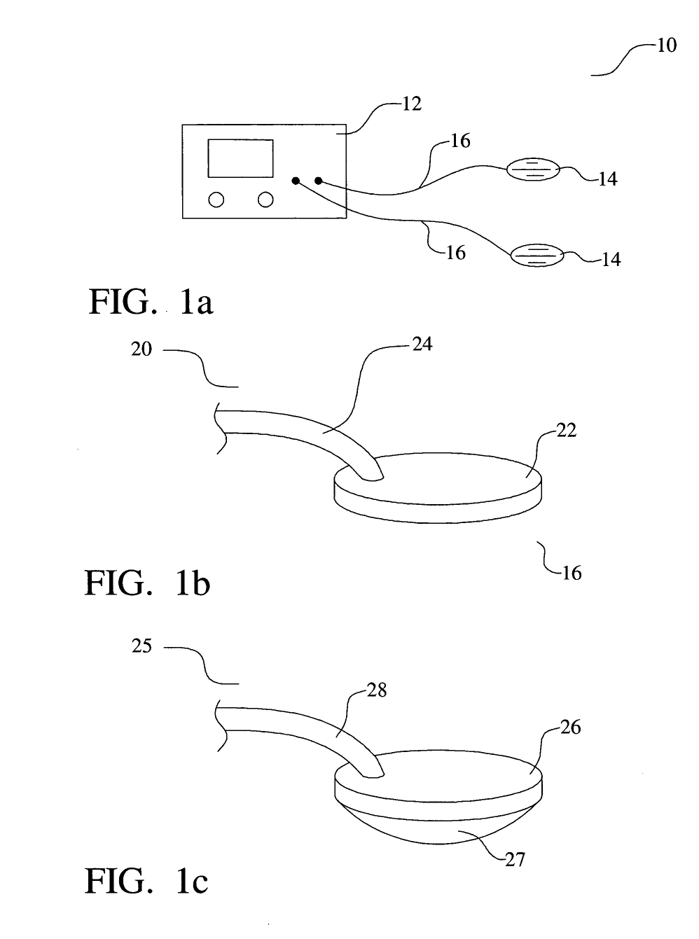

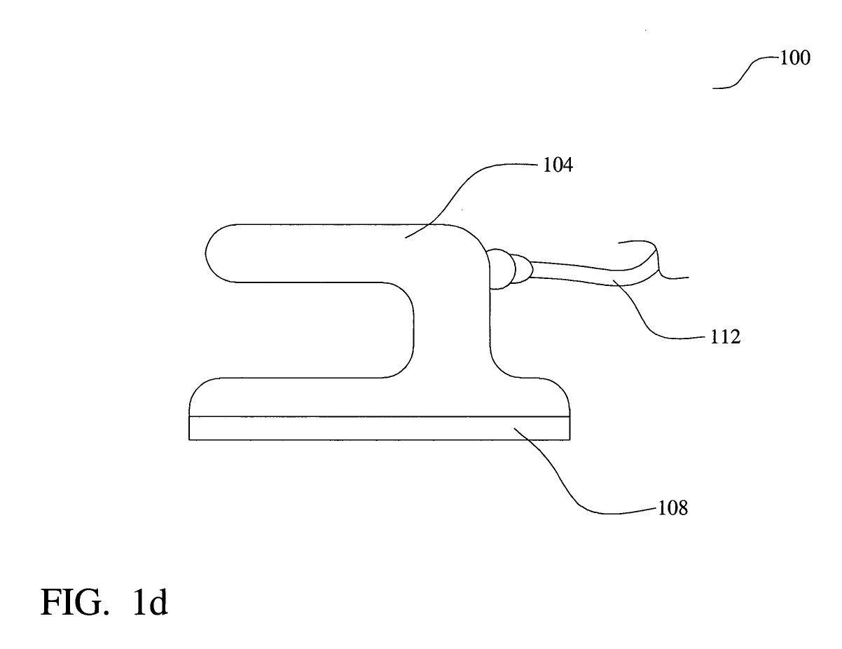

Referring now to FIGS. 1a and 1d, several preferred embodiments of the present invention is illustrated. Several important features of the present invention are shown and discussed below. Referring particularly to FIG. 1a, an electrical stimulation device 10 is shown as a first preferred embodiment. The electrical stimulation device 10 is useful for a variety of medical applications. For example, the stimulation device 10 can be used for heart pacing or defibrillation, nerve stimulation therapy, electrophysiotherapy, and the like. The embodiment comprises an electrical source 12 capable of generating voltage signals of optimal amplitudes, durations, and frequencies. The electrical source preferably comprises a means to generate a voltage signal in the desired range for medical stimulation. These medicinal voltage signals range from very low energy levels for nerve or muscle stimulators up to large energy levels for heart defibrillators. The electrical source provides the stimulus signals at the required voltage amplitude, power capability, frequency, and duration under the control of the medical physician, nurse, or technician.

Transmission of the electrical signal into the patient is performed by connecting conductive paddles 14 to the electrical source 12 through conductive cables 16. The conductive paddles 14, or pads, are then secured to the patient's body. In a nerve or muscle stimulation scenario, a low energy signal is transmitted from the electrical source 12 through the paddles 14 and into the patient's skin. In a defibrillation scenario, a high energy pulse, or series of pulses, is transmitted through the paddles 14 and into the patient's skin in the chest region to re-establish proper heart rhythm, or beating. This high energy pulse is capable of briefly overwhelming the body's electrical system and of ‘jump starting’ the heart back into a proper rhythm.

In one embodiment of the present invention, the conductive paddles 14, or pads, comprise the conductive loaded resin-based material according to the present invention. In this configuration, the paddles 14 preferably comprises a flexible base resin material to thereby optimally fit the contour where applied to the patient's body. In this way, the flexible paddle 14 maximizes the contact area with the body and, therefore, the area of energy transfer. The conductive loaded resin-based paddle 14 has several distinct advantages. First, a flexible and comfortable paddle 14 is fabricated from a medical grade resin. Second, the paddle 14 exhibits low resistivity due to the network of conductive fibers and is, therefore, capable of transferring significant electrical to the patient. Third, the conductive network in the polymeric matrix has a large frequency bandwidth such that rapid switching pulses and/or high frequency signals may be transmitted with little loss. Fourth, the resistivity of the conductive loaded resin-based material allows effective resistance values to be molded into the paddle design 14 for power limiting. Fifth, the paddles are easily formed using resin molding techniques such as injection molding or extrusion to facilitate ease of manufacture and low cost. Sixth, the conductive loaded resin-based electrodes will spread out, or dissipate, the current better than prior art, wire-based electrodes due to the novel conductive matrix. As a result, a larger energy transfer is possible while reducing the occurrence of burning or discomfort.

The present invention may be extended, according to another embodiment, to the formation of a heart pace maker device wherein the paddles and/or the connective cables are placed inside of a patient's body. An FDA approved resin is used for the base resin of the conductive loaded resin-based material.

Referring now to FIG. 1b, in a second preferred embodiment 20 of the present invention, the conductive paddles 22 are connected to the energy source via conductive cables 24. In one embodiment, both the conductive cables 24 and the paddles 22 comprise the conductive loaded resin-based material of the present invention. The low resistivity of the conductive loaded resin-based network allows the electrical signal to be transmitted through the cables 24 with little loss. Further, in another embodiment, the cables 24 and the paddles 22 are molded as a single unit. In yet another embodiment, the cables 24 and the paddles 22 are molded separately and then joined together using, for example, an ultrasonic welding process. The conductive loaded resin-based cable 24 is preferably covered with a non-conductive material such as a non-conductive base resin material.

Alternatively, the cables 24 may comprise a more traditional conductor such as a metal wire or group of wires. In this case, the metal cable 24 may be connected to the paddle 22 by crimping, punching a hole, stapling, and the like. More preferably, the metal cable 24 is connected by soldering. In one embodiment, a hole or perforation is made in the paddle 22. This hole may be molded into the paddle 22 or may be formed by drilling or stamping the paddle after molding. A solderable metal layer, not shown, is then deposited to line or to fill the hole. This solderable layer may comprise a tin-based material or may comprise any other material that is readily solderable. This solderable layer connects into the network of conductive fibers of the conductive loaded resin-based material 22. The cable 24 is then soldered onto the paddle 22 by point soldering, wavesoldering, or reflow.

If a solderable metal layer is used, it may be formed by plating or by coating. If the method of formation is metal plating, then the resin-based structural material of the conductive loaded, resin-based material is one that can be metal plated. There are many of the polymer resins that can be plated with metal layers. For example, GE Plastics, SUPEC, VALOX, ULTEM, CYCOLAC, UGIKRAL, STYRON, CYCOLOY are a few resin-based materials that can be metal plated. The metal layer may be formed by, for example, electroplating or physical vapor deposition.

Referring now to FIG. 1c, a third preferred embodiment of the present invention is illustrated. In this embodiment 25, a gel-like layer 27 is added to the conductive loaded resin-based paddle 26 and cable 28. The gel-like layer 27 comprises a material, such as silicone, that readily conforms to, and adheres to, the skin contour of the patient. Referring now to FIG. 1d, a fourth preferred embodiment of the present invention is illustrated. In this embodiment 100, a paddle with a handle is shown. The operating paddle 108 that contacts the patient's body again comprises the conductive loaded resin-based material of the present invention. In this case, an insulating handle 104 is added attached to the paddle 108 to provide a means for a heath care professional to hold the paddle 108 onto the patient's body without risk of joining the electrical circuit path. Such a procedure is particularly useful for emergency heart resuscitation situations where the defibrillator must be applied quickly. In one embodiment, the handle 104 comprises the same resin as is used as the base resin material for the conductive paddle 108 to provide optimal bonding between the components. The handle 104 is easily two-shot molded onto the paddle 108 or ultrasonically welded onto the paddle 108. In one embodiment, the cable 112 comprises the conductive loaded resin-based material with an insulating layer extruded or coated thereon.

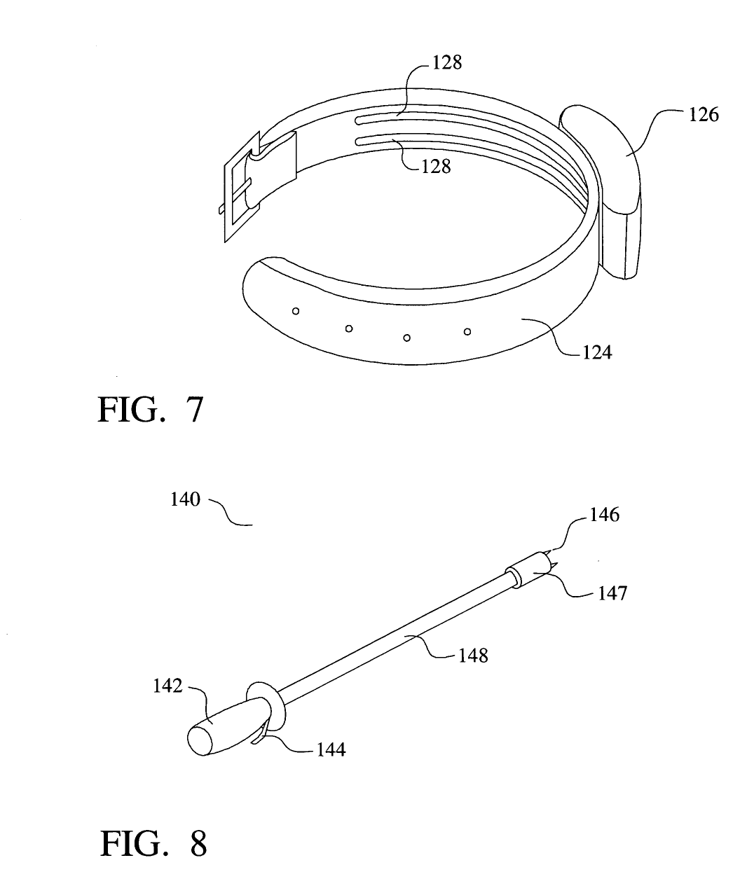

Referring now to FIG. 7, a fifth preferred embodiment of the present invention is illustrated. In this embodiment 120, an animal training or control device 120 is shown. An animal collar, such as could be easily clasped around the neck of a livestock animal or of a pet, is formed, in part, of the conductive loaded resin-based material. In the illustrative embodiment 120, a flexible collar 124 is formed with an attached battery/control pack 126. The battery/control pack 126 is designed to provide a low powered, though uncomfortable, electrical shock to the animal based on contextual control parameters such as the animal's relative position. In the present invention, the battery/control pack 126 is further connected to electrodes 128 embedded in the collar. The electrodes 128 deliver the electrical energy.to the animal.

In one embodiment, the electrodes 128 comprise the conductive loaded resin-based material of the present invention. In one embodiment, the electrodes 128 are continuous strips, as shown. In another embodiment, the electrodes 128 are a series of studs or knobs. Preferably, the collar structure 124 comprises a flexible resin material that is non-conductive and the electrodes 128 comprise the conductive loaded resin-based material using the same flexible base resin mixed with the conductive loading. In another embodiment, the collar structure 124 is a resin material that is over-molded onto the conductive loaded resin-based electrodes 128. In one embodiment the housing of the battery/control pack 126 and the collar structure 124 are formed together by injection molding an insulating resin material. In another embodiment, the electrical circuit within the battery/control pack 126 is connected to the electrodes 128 by soldering, as described above. In another embodiment, the electrical circuit within the battery/control pack 126 comprises conductive loaded resin-based material that is formed together with the electrodes 128 by injection molding.

Referring now to FIG. 8, a sixth preferred embodiment 140 of the present invention is illustrated. In this case, a cattle prod device 140 is formed of the conductive loaded resin-based material. In this case, the electrodes 146 of the prod 140 are formed of the conductive loaded resin-based material. The unique network of micron conductive fibers and/or powders in the polymeric base provides multiple points on the surface of each electrode 146 for electrical charge to exit the electrode 146, to enter the animal's body, and to then be discharged to the ground on which the animal stands. In one embodiment, a battery/control pack is housed within the handle 142. Activation of the prod trigger 144 generates a high voltage potential in the circuit that is transmitted through the shaft 148 to the prod electrodes 146.

In one embodiment, conductive circuit lines between the battery/control pack in the handle 142 and the electrodes 146 also comprises the conductive loaded resin-based material. In this case, the conductive circuit lines are preferably formed together with the electrodes 146 by injection molding or by extrusion molding the conductive loaded resin-based material. Preferably, the handle 142, trigger 144, shaft 148, and electrode head 147 all comprise insulating materials. More preferably, these components comprise insulating resin based materials. In another preferred embodiment, the handle 142, shaft 148, and electrode head 147 are two-shot molded onto conductive loaded resin-based circuit components such as the electrodes 146.

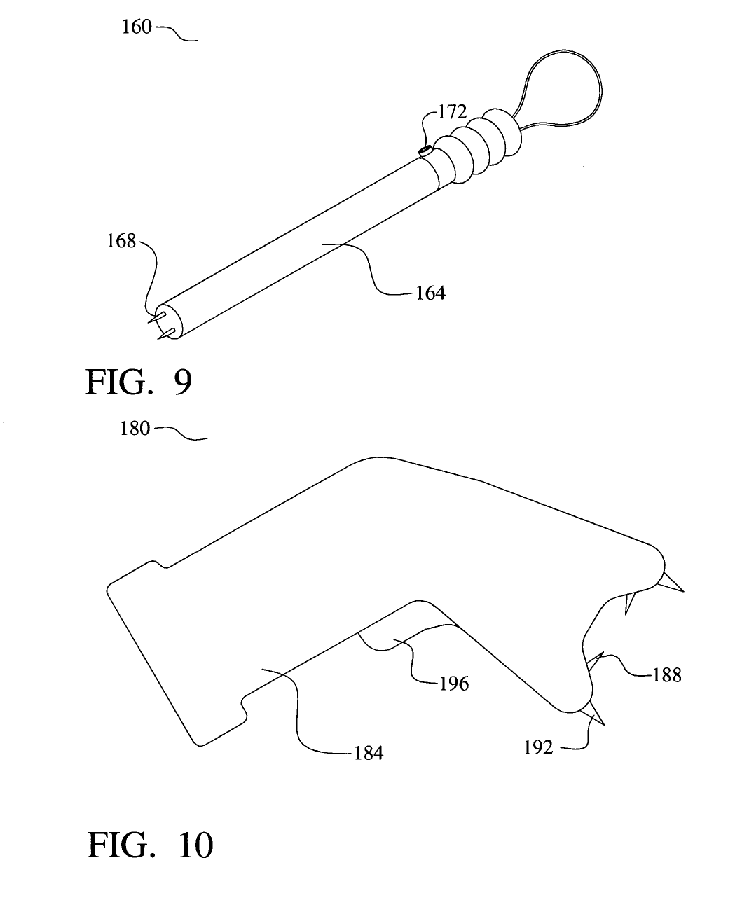

Referring now to FIG. 9, a seventh preferred embodiment of the present invention is illustrated. In this embodiment, a police mechanical/electric baton 160 is shown. The baton 160 incorporates the mechanical, or blunt force, capability of a traditional baton with an electrical stun gun capability. In the preferred embodiment, electrodes 168 comprises the conductive loaded resin-based material of the present invention. The electrodes 168 are connected to a battery/controller pack housed within the baton 160 and more preferably within the handle. When the trigger 172 is activated, a high voltage is generated and propagated to the terminals 168. In another embodiment, the electrical circuit from the internal battery/controller pack and the electrodes 168 also comprises the conductive loaded resin-based material.

The barrel 164 of the baton comprises a hard, insulating material. In one embodiment, the barrel 164 comprises a resin material that is over-molded onto the electrodes 168 and the conductive lines between the electrodes 168 and the battery/controller pack. In a more preferred embodiment, the barrel comprises the same resin material as is used in for the base resin of the conductive loaded resin-based material of the electrodes 168. The novel baton combines the traditional blunt force capability of the night stick with an electrical stun capability. The electrodes 168 discharge energy into a person and then this energy dissipates to the ground on which the person is standing. Depending on the voltage level of the battery/controller pack, the resulting electrical shock can cause anything from a discomfort to pain to temporary loss of motor control.

Referring now to FIG. 10, an eighth preferred embodiment of the present invention is illustrated. In this embodiment, a stun gun 180 is illustrated. As in the case of the baton, the stun gun 180 comprises electrodes 188 and 192 formed of the conductive loaded resin-based material. The stun gun further comprises a battery/controller pack housed within the gun case and, more preferably, within the gun handle 184. When the trigger 196 is depressed, a high voltage is generated within the gun 180 and transmitted across the outer electrodes 192. When the stun electrodes 192 are placed in contact with a person, then this voltage is discharged through the person's body to ground causing the stun effect. As an added feature, the two interior electrodes 188 are placed in close proximity and angled towards each other. In a ‘warning mode’, the stun gun 180 will generate a large voltage potential between the inner electrodes 188. This voltage is substantial enough to cause a very visible and audible electrical arc to be displayed at the end of the gun. This warning arc results in a significant psychological deterrent for an aggressive assailant. In addition, the psychological effect of seeing the aggressive arcing at the electrodes 188 is a very useful means of multiplying the physiological effect when applying the stun gun 180 to a person.

In one embodiment, the internal conductive path from the battery/controller pack to the electrodes 188 and 192 comprises the conductive loaded resin-based material. In this case, both the internal conductive path and the electrodes 188 and 192 are preferably molded together. In another embodiment, the gun case/handle comprise an insulating material and, more preferably, comprise a resin based material that is over-molded onto the internal conductive path and electrodes 188 and 192. In yet another embodiment, the case/handle comprise the same resin material as is used in the conductive loaded resin-based material of the electrodes 188 and 192.

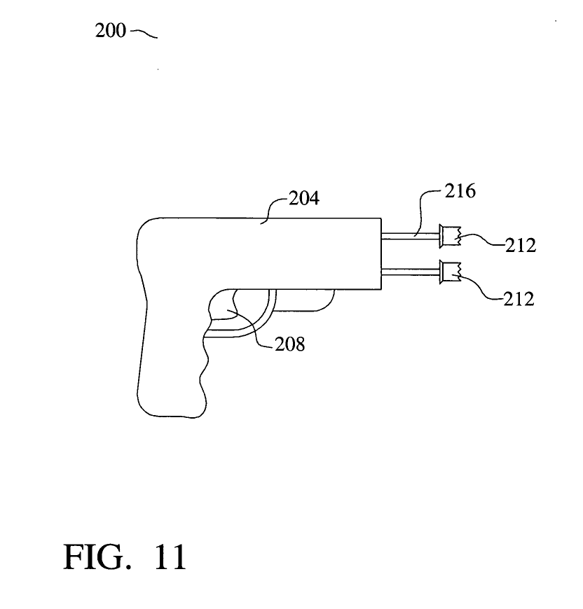

Referring now to FIG. 11, a ninth preferred embodiment of the present invention is illustrated. In this embodiment, a Taser stun gun 200 is illustrated. As in the case of the stun gun 180 above, the electrodes 212 are formed of the conductive loaded resin-based material. However, in this case, the electrodes are shot, or propelled, from the gun to directly contact the assailant from a distance. When the trigger 208 is activated, an air charge is released or chemical charge is detonated such that the terminals 212 are propelled out of the barrel of the gun 200 in the direction that the gun is pointed. As the electrodes 212 travel out of the gun 200, conductive lines 216 that are connected to the electrodes 212 are trailed out from an internal reel. When the electrodes 212 strike the assailant, pins or tins or hooks on the electrodes 212 embed into the person's clothing or skin. Electrical energy from the battery/controller pack housed within the gun case 204 is then transmitted down the conductive lines 216 to be released by the electrodes 212 into the assailant's body.

In one embodiment, the conductive lines 216 from the battery/controller pack to the electrodes 212 comprise the conductive loaded resin-based material. In this case, both the conductive lines and the electrodes 212 are preferably molded together. In another embodiment, the gun case 204 comprises an insulating material. In yet another embodiment, the case 204 comprises the same resin material as is used in the conductive loaded resin-based material of the electrodes 212.

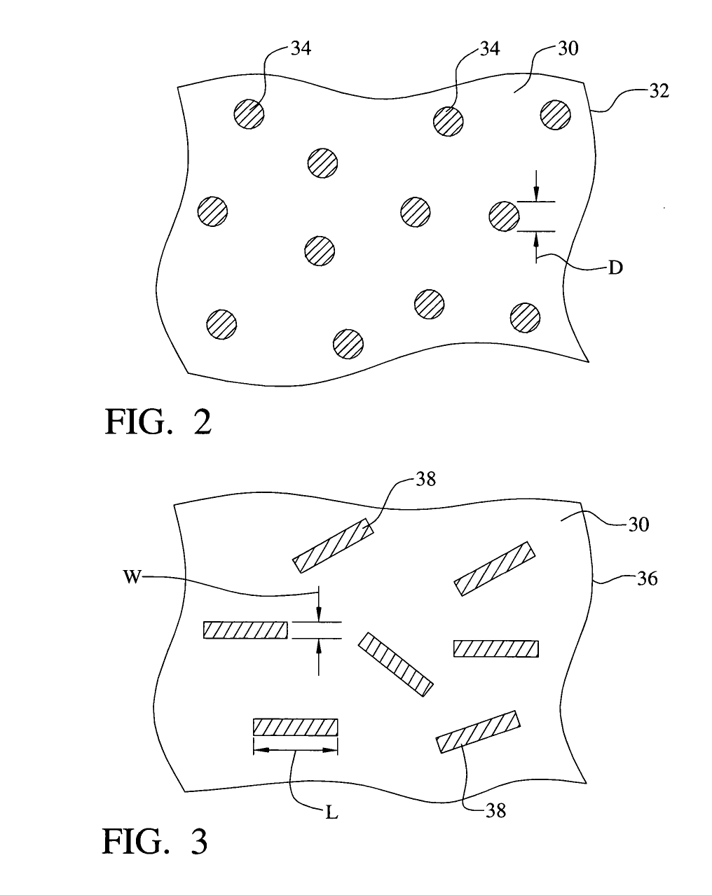

The conductive loaded resin-based material typically comprises a micron powder(s) of conductor particles and/or in combination of micron fiber(s) homogenized within a base resin host. FIG. 2 shows cross section view of an example of conductor loaded resin-based material 32 having powder of conductor particles 34 in a base resin host 30. In this example the diameter D of the conductor particles 34 in the powder is between about 3 and 12 microns.

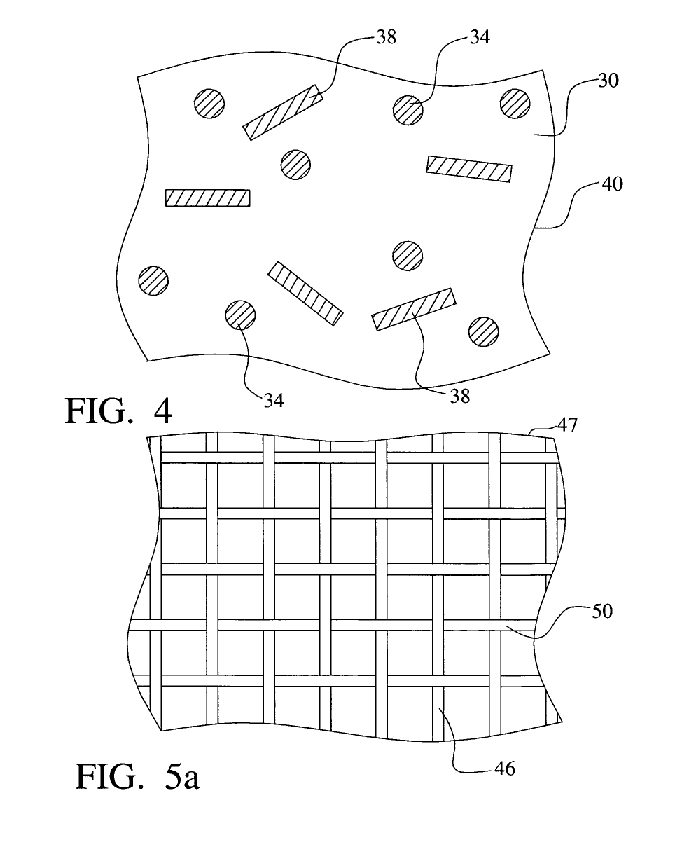

FIG. 3 shows a cross section view of an example of conductor loaded resin-based material 36 having conductor fibers 38 in a base resin host 30. The conductor fibers 38 have a diameter of between about 3 and 12 microns, typically in the range of 10 microns or between about 8 and 12 microns, and a length of between about 2 and 14 millimeters. The conductors used for these conductor particles 34 or conductor fibers 38 can be stainless steel, nickel, copper, silver, or other suitable metals or conductive fibers, or combinations thereof. These conductor particles and or fibers are homogenized within a base resin. As previously mentioned, the conductive loaded resin-based materials have a sheet resistance between about 5 and 25 ohms per square, though other values can be achieved by varying the doping parameters and/or resin selection. To realize this sheet resistance the weight of the conductor material comprises between about 20% and about 50% of the total weight of the conductive loaded resin-based material. More preferably, the weight of the conductive material comprises between about 20% and about 40% of the total weight of the conductive loaded resin-based material. More preferably yet, the weight of the conductive material comprises between about 25% and about 35% of the total weight of the conductive loaded resin-based material. Still more preferably yet, the weight of the conductive material comprises about 30% of the total weight of the conductive loaded resin-based material. Stainless Steel Fiber of 8-11 micron in diameter and lengths of 4-6 mm and comprising, by weight, about 30% of the total weight of the conductive loaded resin-based material will produce a very highly conductive parameter, efficient within any EMF spectrum. Referring now to FIG. 4, another preferred embodiment of the present invention is illustrated where the conductive materials comprise a combination of both conductive powders 34 and micron conductive fibers 38 homogenized together within the resin base 30 during a molding process.

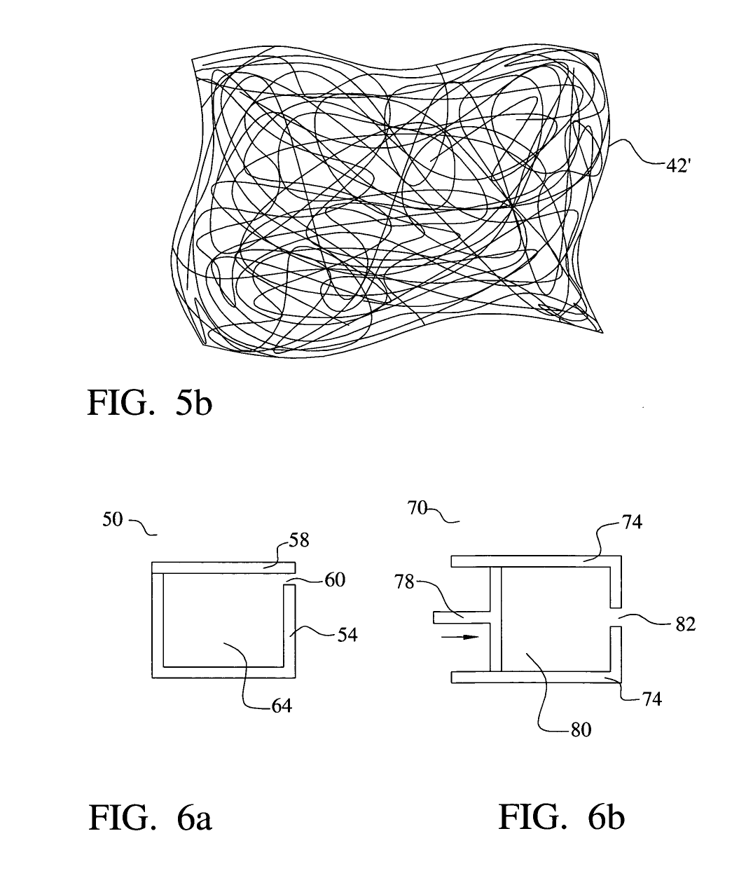

Referring now to FIGS. 5a and 5b, a preferred composition of the conductive loaded, resin-based material is illustrated. The conductive loaded resin-based material can be formed into fibers or textiles that are then woven or webbed into a conductive fabric. The conductive loaded resin-based material is formed in strands that can be woven as shown. FIG. 5a shows a conductive fabric 42 where the fibers are woven together in a two-dimensional weave 46 and 50 of fibers or textiles. FIG. 5b shows a conductive fabric 42′ where the fibers are formed in a webbed arrangement. In the webbed arrangement, one or more continuous strands of the conductive fiber are nested in a random fashion. The resulting conductive fabrics or textiles 42, see FIG. 5a, and 42′, see FIG. 5b, can be made very thin, thick, rigid, flexible or in solid form(s).

Similarly, a conductive, but cloth-like, material can be formed using woven or webbed micron stainless steel fibers, or other micron conductive fibers. These woven or webbed conductive cloths could also be sandwich laminated to one or more layers of materials such as Polyester(s), Teflon(s), Kevlar(s) or any other desired resin-based material(s). This conductive fabric may then be cut into desired shapes and sizes.

Electrical shock, stimulation, or stun devices formed from conductive loaded resin-based materials can be formed or molded in a number of different ways including injection molding, extrusion or chemically induced molding or forming. FIG. 6a shows a simplified schematic diagram of an injection mold showing a lower portion 54 and upper portion 58 of the mold 50. Conductive loaded blended resin-based material is injected into the mold cavity 64 through an injection opening 60 and then the homogenized conductive material cures by thermal reaction. The upper portion 58 and lower portion 54 of the mold are then separated or parted and the electrical shock, stimulation, or stun component and/or device is removed.

FIG. 6b shows a simplified schematic diagram of an extruder 70 for forming electrical shock, stimulation, or stun devices using extrusion. Conductive loaded resin-based material(s) is placed in the hopper 80 of the extrusion unit 74. A piston, screw, press or other means 78 is then used to force the thermally molten or a chemically induced curing conductive loaded resin-based material through an extrusion opening 82 which shapes the thermally molten curing or chemically induced cured conductive loaded resin-based material to the desired shape. The conductive loaded resin-based material is then fully cured by chemical reaction or thermal reaction to a hardened or pliable state and is ready for use. Thermoplastic or thermosetting resin-based materials and associated processes may be used in molding the conductive loaded resin-based articles of the present invention.

The advantages of the present invention may now be summarized. SUMMARIZE OBJECTS.

As shown in the preferred embodiments, the novel methods and devices of the present invention provide an effective and manufacturable alternative to the prior art.

While the invention has been particularly shown and described with reference to the preferred embodiments thereof, it will be understood by those skilled in the art that various changes in form and details may be made without departing from the spirit and scope of the invention.

1. An electrical stimulation device comprising:
an electrical energy source, and
an electrode electrically connected to said electrical energy source wherein said electrode comprises a conductive loaded, resin-based material comprising conductive materials in a base resin host.

2. The device according to claim 1 wherein the percent by weight of said conductive materials is between about 20% and about 50% of the total weight of said conductive loaded resin-based material.

3. The device according to claim 1 wherein the percent by weight of said conductive materials is between about 20% and about 40% of the total weight of said conductive loaded resin-based material.

4. The device according to claim 1 wherein the percent by weight of said conductive materials is between about 25% and about 35% of the total weight of said conductive loaded resin-based material.

5. The device according to claim 1 wherein said conductive materials comprise metal powder.

6. The device according to claim 5 wherein said metal powder is nickel, copper, or silver.

7. The device according to claim 5 wherein said metal powder is a non-conductive material with a metal plating.

8. The device according to claim 7 wherein said metal plating is nickel, copper, silver, or alloys thereof.

9. The device according to claim 5 wherein said metal powder comprises a diameter of between about 3 μm and about 12 μm.

10. The device according to claim 1 wherein said conductive materials comprise non-metal powder.

11. The device according to claim 10 wherein said non-metal powder is carbon, graphite, or an amine-based material.

12. The device according to claim 1 wherein said conductive materials comprise a combination of metal powder and non-metal powder.

13. The device according to claim 1 wherein said conductive materials comprise micron conductive fiber.

14. The device according to claim 13 wherein said micron conductive fiber is nickel plated carbon fiber, or stainless steel fiber, or copper fiber, or silver fiber or combinations thereof.

15. The device according to claim 13 wherein said micron conductive fiber has a diameter of between about 3 μm and about 12 μm and a length of between about 2 mm and about 14 mm.

16. The device according to claim 13 wherein the percent by weight of said micron conductive fiber is between about 20% and about 40% of the total weight of said conductive loaded resin-based material.

17. The device according to claim 13 wherein said micron conductive fiber is stainless steel and wherein the percent by weight of said stainless steel fiber is between about 20% and about 40% of the total weight of said conductive loaded resin-based material.

18. The device according to claim 17 wherein said stainless steel fiber has a diameter of between about 3 μm and about 12 μm and a length of between about 2 mm and about 14 mm.

19. The device according to claim 1 wherein said conductive materials comprise a combination of conductive powder and conductive fiber.

20. The device according to claim 19 wherein said conductive fiber is stainless steel.

21. The device according to claim 1 wherein said base resin and said conductive materials comprise flame-retardant materials.

22. The device according to claim 1 further comprising a metal layer overlying said conductive loaded resin-based material.

23. The device according to claim 1 further comprising a conductive path between said electrical energy source and said electrode wherein said conductive path comprises said conductive loaded resin-based material.

24. The device according to claim 23 wherein said conductive path is a flexible cable.

25. The device according to claim 23 wherein said conductive path is a rigid rod.

26. The device according to claim 1 further comprising a second electrode comprising said conductive loaded resin-based material.

27. The device according to claim 1 further comprising an insulating handle mechanically attached to said electrode.

28. The device according to claim 1 further comprising a gel-like material mechanically attached to said electrode.

29. The device according to claim 1 further comprising a means to propel said electrode away from said electrical energy source.

30. The device according to claim 1 further comprising an enclosure mechanically holding said electrical energy source and said electrode.

31. The device according to claim 30 wherein said enclosure comprises the same resin as is used in said conductive loaded resin based material.

32. An electrical stimulation device comprising:
an electrical energy source,
an electrode comprising a conductive loaded, resin-based material comprising conductive materials in a base resin host; and
a conductive path between said electrical energy source and said electrode wherein said conductive path comprises said conductive loaded resin-based material.

33. The device according to claim 32 wherein the percent by weight of said conductive materials is between about 20% and about 50% of the total weight of said conductive loaded resin-based material.

34. The device according to claim 32 wherein the percent by weight of said conductive materials is between about 20% and about 40% of the total weight of said conductive loaded resin-based material.

35. The device according to claim 32 wherein the percent by weight of said conductive materials is between about 25% and about 35% of the total weight of said conductive loaded resin-based material.

36. The device according to claim 32 wherein said conductive materials comprise metal powder.

37. The device according to claim 36 wherein said metal powder is a non-conductive material with a metal plating.

38. The device according to claim 32 wherein said conductive materials comprise non-metal powder.

39. The device according to claim 32 wherein said conductive materials comprise a combination of metal powder and non-metal powder.

40. The device according to claim 32 wherein said conductive materials comprise micron conductive fiber.

41. The device according to claim 40 wherein the percent by weight of said micron conductive fiber is between about 20% and about 40% of the total weight of said conductive loaded resin-based material.

42. The device according to claim 40 wherein said micron conductive fiber is stainless steel and wherein the percent by weight of said stainless steel fiber is between about 20% and about 40% of the total weight of said conductive loaded resin-based material.

43. The device according to claim 32 wherein said conductive materials comprise a combination of conductive powder and conductive fiber.

44. The device according to claim 32 wherein said conductive fiber is stainless steel.

45. The device according to claim 32 further comprising a metal layer overlying said conductive loaded resin-based material.

46. The device according to claim 32 wherein said conductive path is a flexible cable.

47. The device according to claim 32 wherein said conductive path is a rigid rod.

48. The device according to claim 32 further comprising a second electrode comprising said conductive loaded resin-based material.

49. The device according to claim 32 further comprising an insulating handle mechanically attached to said electrode.

50. The device according to claim 32 further comprising a gel-like material mechanically attached to said electrode.

51. The device according to claim 32 further comprising a means to propel said electrode away from said electrical energy source.

52. The device according to claim 32 further comprising an enclosure mechanically holding said electrical energy source and said electrode.

53. The device according to claim 52 wherein said enclosure comprises the same resin as is used in said conductive loaded resin based material.

54. A method to form an electrode for an electrical stimulation device, said method comprising:
providing a conductive loaded, resin-based material comprising conductive materials in a resin-based host; and
molding said conductive loaded, resin-based material into an electrode.

55. The method according to claim 54 wherein the percent by weight of said conductive materials is between about 20% and about 40% of the total weight of said conductive loaded resin-based material.

56. The method according to claim 54 wherein said conductive materials comprise micron conductive fiber.

57. The method according to claim 56 wherein said micron conductive fiber is nickel plated carbon fiber, or stainless steel fiber, or copper fiber, or silver fiber or combinations thereof.

58. The method according to claim 56 wherein said micron conductive fiber has a diameter of between about 3 μm and about 12 μm and a length of between about 2 mm and about 14 mm.

59. The method according to claim 56 wherein the percent by weight of said micron conductive fiber is between about 20% and about 40% of the total weight of said conductive loaded resin-based material.

60. The method according to claim 56 wherein said micron conductive fiber is stainless steel and wherein the percent by weight of said stainless steel fiber is between about 20% and about 40% of the total weight of said conductive loaded resin-based material.

61. The method according to claim 60 wherein said stainless steel fiber has a diameter of between about 3 μm and about 12 μm and a length of between about 2 mm and about 14 mm.

62. The method according to claim 54 wherein said conductive materials comprise conductive powder.

63. The method according to claim 54 wherein said conductive materials comprise a combination of conductive powder and conductive fiber.

64. The method according to claim 54 wherein said molding comprises:
injecting said conductive loaded, resin-based material into a mold;
curing said conductive loaded, resin-based material; and
removing said electrode device from said mold.

65. The method according to claim 54 wherein said molding comprises:
loading said conductive loaded, resin-based material into a chamber;
extruding said conductive loaded, resin-based material out of said chamber through a shaping outlet; and
curing said conductive loaded, resin-based material to form said electrode device.

66. The method according to claim 54 further comprising subsequent mechanical processing of said molded conductive loaded, resin-based material.

67. The method according to claim 54 further comprising overlying a layer of metal on said molded conductive loaded, resin-based material.
